(19) United States
(12) Patent Application Publication
(10) Pub. No.: US 2022/0167696 A1
(43) Pub. Date: Jun. 2, 2022

(54) IMPREGNATED GLOVE

(71) Applicant: TAYLOR J. MORAN, Rancho Cucamonga, CA (US)

(72) Inventor: TAYLOR J. MORAN, Rancho Cucamonga, CA (US)

(21) Appl. No.: 17/540,992

(22) Filed: Dec. 2, 2021

Related U.S. Application Data
(60) Provisional application No. 63/120,644, filed on Dec. 2, 2020.

(51) Int. Cl. A41D 19/00; A41D 31/30

(52) U.S. Cl. CPC A41D 19/0006; A41D 31/30

(57) ABSTRACT

An impregnated glove assembly includes: a palm portion; a wrist portion coupled to the palm portion; a finger portion coupled to the palm portion and configured to receive two or more fingers of a user; and a treatment solution impregnated within one or more of the palm portion, the wrist portion and the finger portion.

### RELATED APPLICATION(S)

This application claims the benefit of U.S. Provisional Application No. 63/120,644, filed on 2 Dec. 2020, the entire contents of which are incorporated herein by reference.

### TECHNICAL FIELD

This disclosure relates to gloves and, more particularly, to gloves impregnated with a treatment solution.

### BACKGROUND

Impregnated wipes are in common use today, wherein these impregnated wipes may be impregnated with various solutions (e.g., cleansing solutions, conditioning solutions, etc.). Accordingly and through the use of such impregnated wipes, the process of cleaning objects or people may be expedited.

Unfortunately and in order to use such impregnated wipes, the impregnated wipe must be held by the hand through the use of the thumb, thus limiting the ability to use the individual fingers to providing a higher level of cleaning efficacy.

### SUMMARY OF DISCLOSURE

In one implementation, an impregnated glove assembly includes: a palm portion; a wrist portion coupled to the palm portion; a finger portion coupled to the palm portion and configured to receive two or more fingers of a user; and a treatment solution impregnated within one or more of the palm portion, the wrist portion and the finger portion.

One or more of the following features may be included. The treatment solution may include one or more of: an organic treatment solution; and an inorganic treatment solution. The treatment solution may include or more of: a disinfecting treatment solution; an antimicrobial treatment solution; an antibiotic treatment solution; a cleansing treatment solution; and a conditioning treatment solution. The treatment solution may include one or more natural oils. The one or more natural oils may include one or more of: an oregano oil; a peppermint oil; a lemon oil; a tea tree oil; a lavender oil; an orange oil; a eucalyptus oil; a citronella oil; a thyme oil; a chamomile oil; a marjoram oil; a witch hazel oil; a frankincense oil; a thieves oil; an aloe oil; a cinnamon oil; and a clove oil. The treatment solution may include one or more solvents. The treatment solution may include one or more petrochemicals. The impregnated glove assembly may be constructed, at least in part, of a natural fiber. The natural fiber may include a wood fiber. The natural fiber may include a bamboo fiber. The impregnated glove assembly may be constructed, at least in part, of a synthetic fiber. The synthetic fiber may include a plastic fiber. The finger portion may be configured to receive five fingers. The wrist portion may include one or more removal assistance tabs. The wrist portion may be configured to cinch around a wrist of the user. The impregnated glove assembly may include one or more graphics. The one or more graphics may include one or more child-friendly graphics.

In another implementation, an impregnated glove assembly may include: a palm portion; a wrist portion coupled to the palm portion; a finger portion coupled to the palm portion and configured to receive two or more fingers of a user; and an organic treatment solution impregnated within one or more of the palm portion, the wrist portion and the finger portion.

One or more of the following features may be included. The organic treatment solution may include or more of: a disinfecting treatment solution; an antimicrobial treatment solution; an antibiotic treatment solution; a cleansing treatment solution; and a conditioning treatment solution. The organic treatment solution may include one or more natural oils. The one or more natural oils may include one or more of: an oregano oil; a peppermint oil; a lemon oil; a tea tree oil; a lavender oil; an orange oil; a eucalyptus oil; a citronella oil; a thyme oil; a chamomile oil; a marjoram oil; a witch hazel oil; a frankincense oil; a thieves oil; an aloe oil; a cinnamon oil; and a clove oil. The impregnated glove assembly may be constructed, at least in part, of a natural fiber. The natural fiber may include a wood fiber. The natural fiber may include a bamboo fiber. The wrist portion may be configured to cinch around a wrist of the user.

In another implementation, an impregnated glove assembly includes: a palm portion; a wrist portion coupled to the palm portion and including one or more removal assistance tabs; a finger portion coupled to the palm portion and configured to receive five fingers of a user; and an organic treatment solution impregnated within one or more of the palm portion, the wrist portion and the finger portion, wherein the organic treatment solution includes one or more natural oils; wherein the impregnated glove assembly is constructed, at least in part, of a natural fiber.

One or more of the following features may be included. The organic treatment solution may include or more of: a disinfecting treatment solution; an antimicrobial treatment solution; an antibiotic treatment solution; a cleansing treatment solution; and a conditioning treatment solution. The one or more natural oils may include one or more of: an oregano oil; a peppermint oil; a lemon oil; a tea tree oil; a lavender oil; an orange oil; a eucalyptus oil; a citronella oil; a thyme oil; a chamomile oil; a marjoram oil; a witch hazel oil; a frankincense oil; a thieves oil; an aloe oil; a cinnamon oil; and a clove oil. The natural fiber may include a wood fiber. The natural fiber may include a bamboo fiber.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic view of an impregnated glove assembly according to an embodiment of the present disclosure;

FIG. 1A is a table illustrating the antibacterial/antiviral/antibiotic/disinfectant qualities of various natural oils; and

FIG. 2 is another diagrammatic view of the impregnated glove assembly of FIG. 1 according to an embodiment of the present disclosure.

Like reference symbols in the various drawings indicate like elements.

### DETAILED DESCRIPTION OF THE PREFERRED EMBODIMENTS

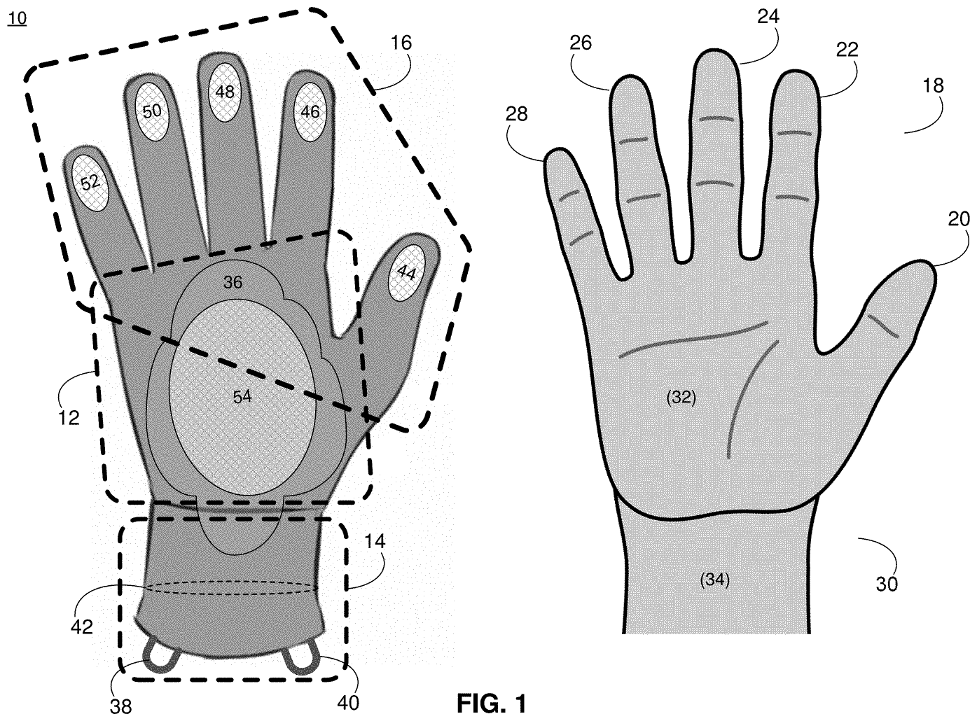

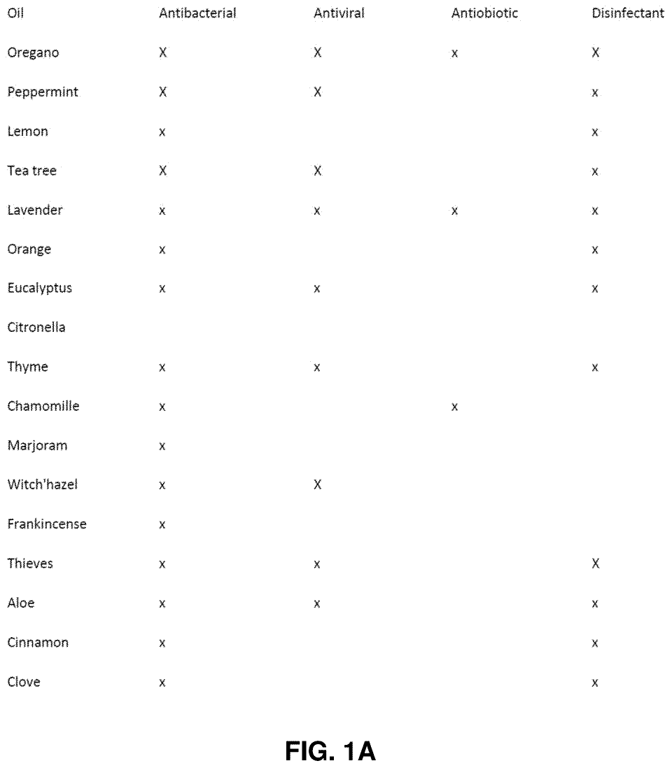

Referring to FIG. 1, there is shown impregnated glove assembly 10. Impregnated glove assembly 10 may include palm portion 12 and wrist portion 14 coupled to palm portion 12.

Impregnated glove assembly 10 may include finger portion 16 coupled to palm portion 12 and configured to receive two or more fingers 18 (e.g., fingers 20, 22, 24, 26, 28) of user 30. For example, finger portion 16 may be configured to receive five fingers (e.g., fingers 20, 22, 24, 26, 28). Accordingly, impregnated glove assembly 10 may be configured to cover the entire hand of user 30; namely fingers 20, 22, 24, 26, 28, palm portion 32 and some or all of wrist 34.

Some or all of impregnated glove assembly 10 may be impregnated (e.g., saturated/infused/moistened) with treatment solution 36. For example, treatment solution 36 may be impregnated within one or more of palm portion 12, wrist portion 14 and finger portion 16.

Examples of treatment solution 36 may include one or more of an organic treatment solution and an inorganic treatment solution.
Organic Treatment Solutions: Examples of an organic treatment solution may include treatment solutions that are e.g., plant-based, sustainable, biodegradable, natural, non-toxic, hypoallergenic, child safe and/or pet safe. Additionally, treatment solution 36 may include one or more natural oils, examples of which may include but are not limited to: an oregano oil; a peppermint oil; a lemon oil; a tea tree oil; a lavender oil; an orange oil; a eucalyptus oil; a citronella oil; a thyme oil; a chamomile oil; a marjoram oil; a witch hazel oil; a frankincense oil; a thieves oil; an aloe oil; a cinnamon oil; and a clove oil. These natural may have different qualities with respect to e.g., antibacterial qualities, antiviral qualities, antibiotic qualities and/or disinfectant qualities (as illustrated in FIG. 1A).
Inorganic Treatment Solutions: Examples of an inorganic treatment solution may include treatment solutions that contain e.g., sulphates (SLS, SLES, SCS), triclosan, sodium coco sulphate, coco betaine, parabens, phosphates, formaldehyde, MEA, DEA, TEA, synthetic fragrance, chlorine, bleach, petrochemicals, solvents, animal by-products, allergens, perfumes, dyes and/or caustic substances.

Treatment solution 36 may include one or more of: a disinfecting treatment solution; an antimicrobial treatment solution; an antibiotic treatment solution; a cleansing treatment solution; and a conditioning treatment solution.
Disinfecting Treatment Solution: A disinfecting treatment solution is generally a substance used to inactivate or destroy microorganisms on inert surfaces.
Antimicrobial Treatment Solution: An antimicrobial treatment solution is generally a substance that is active against bacteria, is used to fight bacterial infections, and may kill or inhibit the growth of bacteria.
Antibiotic Treatment Solution: An antibiotic treatment solution is generally a substance applied to living tissue/skin to reduce the possibility of infection, sepsis, or putrefaction.
Cleansing Treatment Solution: A cleansing treatment solution is generally a substance that cleans or removes dirt or other substances (e.g., such as a detergent). Example may include but are not limited to general purposes cleansers, household cleansers, kitchen cleansers, automotive cleansers, cosmetology cleansers, dermatology cleansers and/or skin cleansers.
Conditioning Treatment Solution: A conditioning treatment solution is generally a substance that improves the quality of another material. Example may include but are not limited to general purposes conditioners, skin conditioners, leather conditioners, vinyl conditioners, plastic conditioners and/or metal conditioners.

Impregnated glove assembly 10 may be constructed, at least in part, of a natural fiber. Examples of such a natural fiber may include but are not limited to a wood fiber and a bamboo fiber.
Wood Fiber: Wood pulp is a lignocellulosic fibrous material prepared by chemically or mechanically separating cellulose fibers from wood, fiber crops, waste paper, or rags. Mixed with water and other chemical or plant-based additives, wood pulp is the major raw material used in papermaking and the industrial production of other paper products.
Bamboo Fiber: Bamboo is one of the best alternatives to use when manufacturing paper, as bamboo is a quickly renewable resource that may be grown on a large scale. Specifically, bamboo is one of the fastest-growing plants, wherein some species may grow more than a meter per day. When compared to trees (which require decades to recover from harvesting), bamboo may require only 3-5 years to reach maturity.

Additionally/alternatively, impregnated glove assembly 10 may be constructed, at least in part, of a synthetic fiber. Such synthetic fiber construction may be done in its entirety or selectively for reinforcement purposes only. An example of such a synthetic fiber may include but is not limited to a plastic fiber.
Plastic Fiber: Polyester is a generalized term for any fabric or textile, which is made using polyester yarns or fibers. The name is shortened from a synthetic, man-made polymer, which is most commonly referred to as polyethylene terephthalate (PET), wherein it may be made by mixing ethylene glycol and terephthalic acid.

Wrist portion 14 may include one or more removal assistance tabs (e.g., removable assistant tabs 38, 40). For example, removal assistance tabs (e.g., removable assistant tabs 38, 40) may stand proud of wrist portion 14 and thus be easily grabbable, thus enabling easy removal of impregnated glove assembly 10 from the hand of user 30 without needing to reach under wrist portion 14 and touch wrist 34 of user 30.

Wrist portion 14 may be configured to cinch around wrist 34 of user 30. For example, wrist portion 14 of impregnated glove assembly 10 may include an elastic type material/structure (e.g., elastic structure 42) to effectuate such cinching around wrist 34 of user 30.

Palm portion 12 and/or finger portion 16 may include one or more scrubbing portions that allow user 30 to scrub whatever surface they are cleaning. For example, scrubbing portions 44, 46, 48, 50, 52 may be affixed to finger portion 16 and may allow user 30 to provide such scrubbing action to such a surface via fingers 20, 22, 24, 26, 28 (respectively). Further, scrubbing portion 54 may be affixed to palm portion 12 and may allow user 30 to provide such scrubbing action to such a surface via palm portion 32. Examples of scrubbing portions 44, 46, 48, 50, 52, 54 may include but are not limited to portions of abrasive material, portions of textured material and/or portions of enhanced friction material.

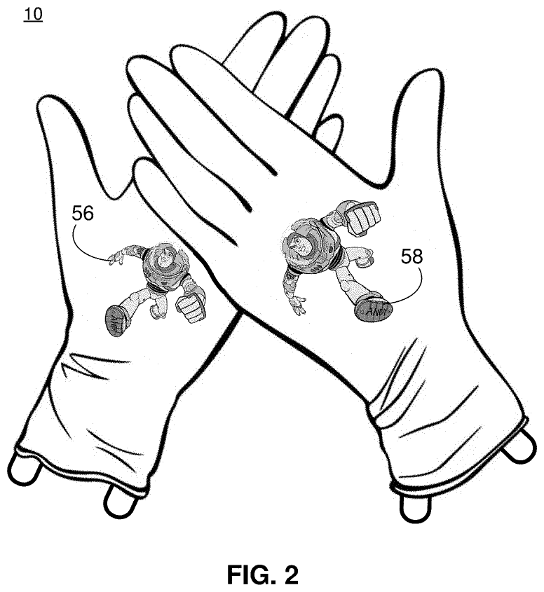

Referring also to FIG. 2, impregnated glove assembly 10 may include one or more graphics (e.g., graphics 56, 58), wherein these one or more graphics (e.g., graphics 56, 58) may include one or more child-friendly graphics. Accordingly and through the use of such graphics (e.g., graphics 56, 58), the cleaning of a child's face after eating a messy food (e.g., ice cream) may be a more kid-friendly/enjoyable experience.

### General

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object oriented programming language such as Java, Smalltalk, C++ or the like. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet (e.g., network 14).

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the flowchart and/or block diagram block or blocks.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. An impregnated glove assembly comprising:
a palm portion;
a wrist portion coupled to the palm portion;
a finger portion coupled to the palm portion and configured to receive two or more fingers of a user; and
a treatment solution impregnated within one or more of the palm portion, the wrist portion and the finger portion.

2. The impregnated glove assembly of claim 1 wherein the treatment solution includes one or more of:
an organic treatment solution; and
an inorganic treatment solution.

3. The impregnated glove assembly of claim 1 wherein the treatment solution includes or more of:
a disinfecting treatment solution;
an antimicrobial treatment solution;
an antibiotic treatment solution;
a cleansing treatment solution; and
a conditioning treatment solution.

4. The impregnated glove assembly of claim 1 wherein the treatment solution includes one or more natural oils.

5. The impregnated glove assembly of claim 4 wherein the one or more natural oils includes one or more of:
an oregano oil;
a peppermint oil;
a lemon oil;
a tea tree oil;
a lavender oil;
an orange oil;
a eucalyptus oil;
a citronella oil;
a thyme oil;
a chamomile oil;
a marjoram oil;
a witch hazel oil;
a frankincense oil;
a thieves oil;
an aloe oil;
a cinnamon oil; and
a clove oil.

6. The impregnated glove assembly of claim 1 wherein the treatment solution includes one or more solvents.

7. The impregnated glove assembly of claim 1 wherein the treatment solution includes one or more petrochemicals.

8. The impregnated glove assembly of claim 1 wherein the impregnated glove assembly is constructed, at least in part, of a natural fiber.

9. The impregnated glove assembly of claim 8 wherein the natural fiber includes a wood fiber.

10. The impregnated glove assembly of claim 8 wherein the natural fiber includes a bamboo fiber.

11. The impregnated glove assembly of claim 1 wherein the impregnated glove assembly is constructed, at least in part, of a synthetic fiber.

12. The impregnated glove assembly of claim 11 wherein the synthetic fiber includes a plastic fiber.

13. The impregnated glove assembly of claim 1 wherein the finger portion is configured to receive five fingers.

14. The impregnated glove assembly of claim 1 wherein the wrist portion includes one or more removal assistance tabs.

15. The impregnated glove assembly of claim 1 wherein the wrist portion is configured to cinch around a wrist of the user.

16. The impregnated glove assembly of claim 1 wherein the impregnated glove assembly includes one or more graphics.

17. The impregnated glove assembly of claim 16 wherein the one or more graphics include one or more child-friendly graphics.

18. An impregnated glove assembly comprising:
a palm portion;
a wrist portion coupled to the palm portion;
a finger portion coupled to the palm portion and configured to receive two or more fingers of a user; and
an organic treatment solution impregnated within one or more of the palm portion, the wrist portion and the finger portion.

19. The impregnated glove assembly of claim 18 wherein the organic treatment solution includes or more of:
a disinfecting treatment solution;
an antimicrobial treatment solution;
an antibiotic treatment solution;
a cleansing treatment solution; and
a conditioning treatment solution.

20. The impregnated glove assembly of claim 18 wherein the organic treatment solution includes one or more natural oils.

21. The impregnated glove assembly of claim 20 wherein the one or more natural oils includes one or more of:
an oregano oil;
a peppermint oil;
a lemon oil;
a tea tree oil;
a lavender oil;
an orange oil;
a eucalyptus oil;
a citronella oil;
a thyme oil;
a chamomile oil;
a marjoram oil;
a witch hazel oil;
a frankincense oil;
a thieves oil;
an aloe oil;
a cinnamon oil; and
a clove oil.

22. The impregnated glove assembly of claim 18 wherein the impregnated glove assembly is constructed, at least in part, of a natural fiber.

23. The impregnated glove assembly of claim 22 wherein the natural fiber includes a wood fiber.

24. The impregnated glove assembly of claim 22 wherein the natural fiber includes a bamboo fiber.

25. The impregnated glove assembly of claim 18 wherein the wrist portion is configured to cinch around a wrist of the user.

26. An impregnated glove assembly comprising:
a palm portion;
a wrist portion coupled to the palm portion and including one or more removal assistance tabs;
a finger portion coupled to the palm portion and configured to receive five fingers of a user; and
an organic treatment solution impregnated within one or more of the palm portion, the wrist portion and the finger portion, wherein the organic treatment solution includes one or more natural oils;
wherein the impregnated glove assembly is constructed, at least in part, of a natural fiber.

27. The impregnated glove assembly of claim 26 wherein the organic treatment solution includes or more of:
a disinfecting treatment solution;
an antimicrobial treatment solution;
an antibiotic treatment solution;
a cleansing treatment solution; and
a conditioning treatment solution.

28. The impregnated glove assembly of claim 26 wherein the one or more natural oils includes one or more of:
an oregano oil;
a peppermint oil;
a lemon oil;
a tea tree oil;
a lavender oil;
an orange oil;
a eucalyptus oil;
a citronella oil;
a thyme oil;
a chamomile oil;
a marjoram oil;
a witch hazel oil;
a frankincense oil;
a thieves oil;
an aloe oil;
a cinnamon oil; and
a clove oil.

29. The impregnated glove assembly of claim 26 wherein the natural fiber includes a wood fiber.

30. The impregnated glove assembly of claim 26 wherein the natural fiber includes a bamboo fiber.
